(19) United States
(12) Patent Application Publication
(10) Pub. No.: US 2019/0015107 A1
(43) Pub. Date: Jan. 17, 2019

(54) Vaso-Occlusive Device and Method

(71) Applicant: Spartan Micro, Inc., Fremont, CA (US)

(72) Inventors: Mark Dias, Round Rock, TX (US); Eric Stoppenhagen, Round Rock, TX (US)

(73) Assignee: Spartan Micro, Inc., Fremont, CA (US)

(21) Appl. No.: 15/649,176

(22) Filed: Jul. 13, 2017

(51) Int. Cl. A61B 17/12

(52) U.S. Cl. CPC A61B 17/1214; A61B 2017/1205; A61B 17/12113; A61B 17/12031

(57) ABSTRACT

Vaso-occlusive coils, and methods of their use, are disclosed having configurations suitable for treating certain problems associated with the vasculature, such as aneurysms, for example. The vaso-occlusive coils described or contemplated herein are configured to improve adherence to aneurysms resulting in diversion of blood flow around the aneurysm while promoting healing of the vasculature.

### BACKGROUND

### Field of this Disclosure

This disclosure generally relates to endovascular devices, and more particularly, to endovascular occlusion devices used in the treatment of an aneurysm.

### Description of the Related Art

Endovascular coiling is often utilized in the treatment of an aneurysm. A microcatheter may be introduced into a patient's vasculature, the tip of the microcatheter further advanced to the site of the aneurysm, for example. Once positioned, one or more vascular occluding or vaso-occlusive coils may then be advanced through the microcatheter and into a void of the aneurysm. Once positioned within the void, the body responds by forming a clot around the coils, reducing the associated blood pressure and risk of rupture of the aneurysm.

During such procedures, one or more of the advancing vaso-occlusive coil structures may exit the aneurysm void, entering the patient's vasculature. The coil must then be retracted and advanced once more, ensuring that the coil remains within the aneurysm. Such additional steps unnecessarily prolong the endovascular procedure, resulting in increased risk to the patient. Additionally, after such procedures, at least a portion of the one or more vaso-occlusive coil structures may exit the void of the aneurysm and enter the patient's vasculature, reducing blood flow in the area.

Accordingly, there is a need for a vascular occluding coil that encourages deployment within the void of an aneurysm.

### BRIEF SUMMARY

Consistent with the present disclosure, a vaso-occlusive coil may have a primary shape when confined within a lumen of a deployment device, and a secondary shape when unconfined, and may include one or more of the following features. The primary shape may be generally straight or curvilinear, e.g., following a shape of a lumen as part of the deployment device, while the secondary shape may include a plurality of geometric shapes. The plurality of geometric shapes may cooperate to form a central void. One or more of the plurality of geometric shapes may have a geometric plane generally along its longitudinal axis, the geometric plane of the one or more of the plurality of geometric shapes may be configured to pass through a portion of the central void. The geometric shape of the one or more of the plurality of geometric shapes may pass through a portion of the central void.

Certain embodiments may include one or more of the following features. The vaso-occlusive coil may be configured to transition from the primary shape to the secondary shape as the vaso-occlusive coil is deployed from a distal opening of the lumen of the deployment device. Each of the plurality of geometric shapes may be selected from a group of shapes including a circular shape, a rectangular shape, and a triangular shape. Each of the plurality of geometric shapes may have a length, the length of a first of the plurality of geometric shapes may be less than a length of each of the remaining ones of the plurality of geometric shapes. A length of a second of the plurality of geometric shapes may be greater than the first of the plurality of geometric shapes, but less than a length of each of the remaining ones of the plurality of geometric shapes.

Certain embodiments may include one or more of the following features. A length of the coil may extend along the plurality of geometric shapes and a first of the plurality of geometric shapes may be a distal-most geometric shape along the length of the coil. The first of the plurality of geometric shapes may be adjacent to a second of the plurality of geometric shapes along the length of the coil. The second of the plurality of geometric shapes may include a geometric plane generally along its longitudinal axis, and the geometric plane of the second of the plurality of geometric shapes may be configured to pass through the central void. A length of the second of the plurality of geometric shapes may be less than a length of each of the remaining ones of the plurality of geometric shapes. A length of the second of the plurality of geometric shapes may be greater than a length of the first of the plurality of geometric shapes. Each of the plurality of geometric shapes may include a respective one of a plurality of geometric planes, generally along its corresponding longitudinal axis, the geometric plane of the first of the plurality of geometric planes being nonparallel to the remaining ones of the plurality of geometric planes. The geometric plane of the first of the plurality of geometric planes may be non-perpendicular to remaining ones of the plurality of geometric planes. Each of the geometric planes of the first and second of the plurality of geometric planes may be nonparallel to the remaining ones of the plurality of geometric planes. Each of the geometric planes of the first and second of the plurality of geometric planes may be non-perpendicular to the remaining ones of the plurality of geometric planes. A portion of the first of the plurality of geometric shapes may pass through the geometric plane of a second of the plurality of geometric shapes, the second of the plurality of geometric shapes being adjacent to the first of the plurality of geometric shapes.

In another aspect, a method may include providing an vaso-occlusive coil having a primary shape and a secondary shape, the secondary shape including a plurality of geometric shapes. A first of the plurality of geometric shapes may have a geometric plane generally along its longitudinal axis. Remaining ones of the plurality of geometric shapes may cooperate to form a central void, and the geometric plane of the first of the plurality of geometric shapes may pass into the central void. The method may include advancing a deployment device to a target tissue site, the deployment device having a lumen there through, and may include advancing the vaso-occlusive coil through the lumen of the deployment device until the vaso-occlusive coil transitions from the primary shape to the secondary shape.

### BRIEF DESCRIPTION OF THE DRAWINGS

Reference will be made to embodiments of the disclosure, examples of which may be illustrated in the accompanying figures. These figures are intended to be illustrative, not limiting. Although certain aspects of the embodiments are generally described, it should be understood that such description is not intended to limit the scope to these particular embodiments. In the drawings:

FIG. 1 depicts a perspective view of an vaso-occlusive coil, in accordance with this disclosure.

FIG. 2 depicts a side elevation view of the vaso-occlusive coil of FIG. 1.

FIG. 3 depicts another side elevation view of the vaso-occlusive coil of FIG. 1.

FIG. 4 depicts lengths of exemplary geometric shapes that may be utilized in an vaso-occlusive coil, in accordance with this disclosure.

FIG. 5 depicts partial deployment of an vaso-occlusive coil, in accordance with this disclosure.

FIG. 6 depicts another partial deployment of an vaso-occlusive coil, in accordance with this disclosure.

FIG. 7A depicts an exemplary method, in accordance with this disclosure.

FIG. 7B depicts detail of a step of the exemplary method of FIG. 7A.

### DETAILED DESCRIPTION

Vaso-occlusive coils, and methods of their use, are disclosed having configurations suitable for treating certain problems associated with the vasculature, such as aneurysms, for example. The vaso-occlusive coils described or contemplated herein are configured to improve adherence to aneurysms resulting in diversion of blood flow around the aneurysm while promoting healing of the vasculature.

The following description is set forth for explanation to provide an understanding of the various embodiments of the present disclosure. However, as should be apparent, one skilled in the art will recognize that embodiments of the present disclosure may be incorporated into numerous other embodiments, devices and systems.

The embodiments of the present disclosure may include certain aspects each of which may be present in one or more medical devices or systems thereof. Assemblies and devices shown below are not necessarily to scale and are illustrative of exemplary embodiments. Furthermore, the illustrated exemplary embodiments disclosed herein may include more, or less, structures than depicted, and are not intended to be limited to the specific depicted structures. While various portions of the present disclosure are described relative to specific structures or processes with respect to a medical device or system using specific labels, these labels are not meant to be limiting.

The vaso-occlusive coils described herein may be made from any suitable biocompatible material, such as wire, including but not limited to metals (e.g., platinum), metal alloys (e.g. stainless steel, Nitinol) and/or polymers (e.g. polycarbonate), and may be formed using any appropriate process. For example, the vaso-occlusive coils described herein may be formed using wire wrapped around one or more mandrels of various sizes and/or further subjecting the wire to heat in order to obtain a desired shape, as is known in the art. The wire may have any desired cross-section, including but not limited to, circular, rectangular, or triangular. For illustrative purposes only, any of the embodiments described or contemplated herein may utilize wire having a cross-sectional dimension in the range of about 0.00002 to about 0.01 inches, and associated coil loops formed by the wire may have a cross-sectional dimension between about 0.003 to about 0.03 inches, and preferably from about 0.009 inches to about 9.915 inches. Furthermore, for illustration purposes only, the vaso-occlusive coils described or contemplated herein may have an axial length in the range of about 0.2 inches to about 40 inches, and may have from 20 to 150 turns per inch.

Reference will now be made in detail to the present exemplary embodiments, which are illustrated in the accompanying drawings.

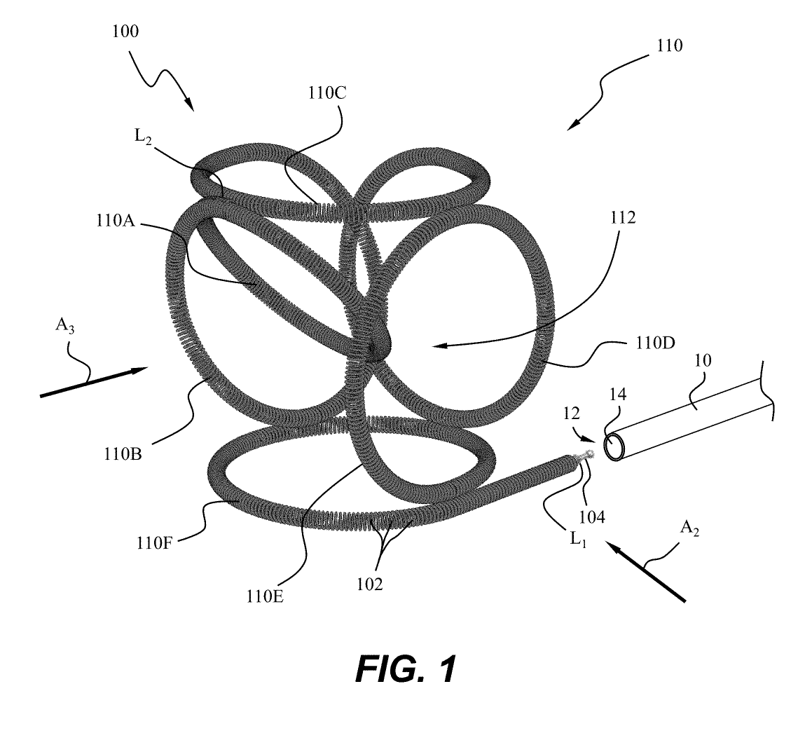

Turning to FIG. 1, a vaso-occlusive coil 100 in accordance with this disclosure is depicted after deployment from a catheter 10, as part of a deployment system. The catheter 10 may include a lumen 12 ending in a distal opening 14. The coil 100 may have a primary shape when positioned within the lumen 12 of the catheter 10, e.g., generally taking on the shape of the lumen 12 of catheter 10, and a secondary shape once fully deployed from the distal opening 14 and unconfined, as generally depicted in FIG. 1. The coil 100 may include individual coils 102 ending in an engaging member 104 configured to interface with the deployment system (not shown). The deployment system may be configured to control advancement of the coil 100 through the lumen 14, and out the distal opening 12, of the catheter 10. Any suitable deployment system and corresponding engaging member suitable to detach the vaso-occlusive coil 100 from the deployment system may be utilized.

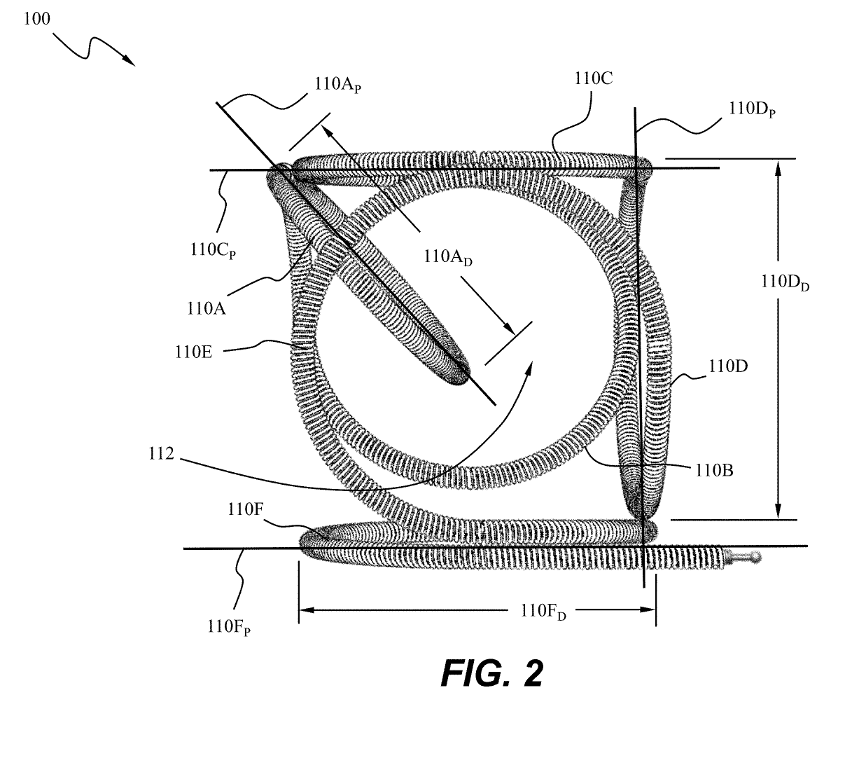

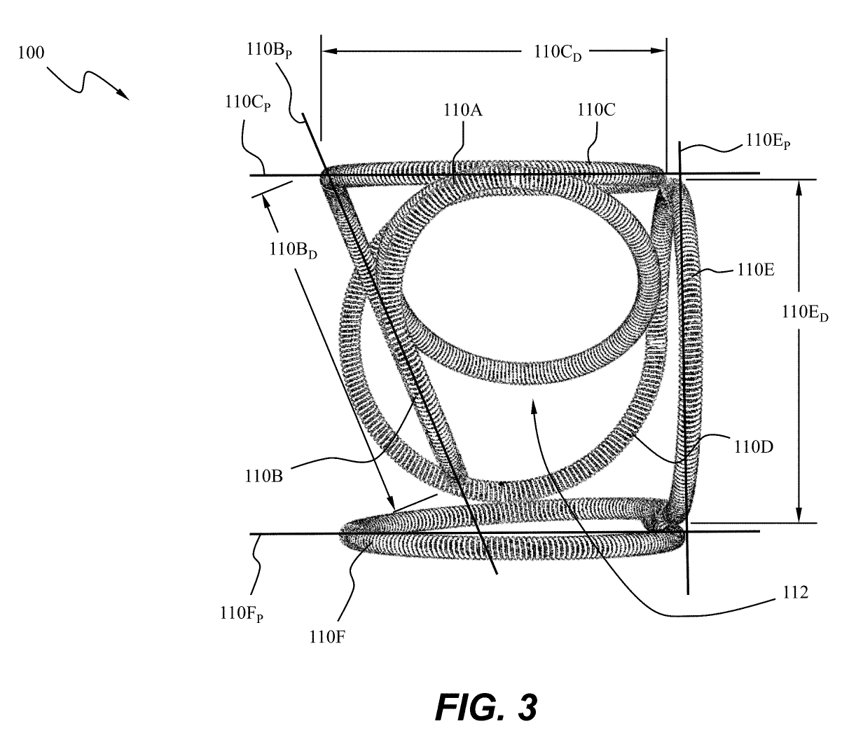

When fully deployed, as depicted in FIG. 1, the vaso-occlusive coil 100 has an axial length, L, extending along a longitudinal axis of the coil 100, from a proximal position L₁ to a distal position L₂. For illustration purposes only, the overall length L may be in the range from about 0.2 inches to about 40 inches. When deployed, the vaso-occlusive coil 100 may include a plurality of geometric shapes, collectively referred to as geometric shapes 110, and individually referred to as geometric shapes 110A-110F, geometric shape 110A being a distal-most geometric shape along the length L of the coil 100 and geometric shape 110F being a proximal most geometric shape along the length L of the coil 100. Each of the plurality of geometric shapes 110 may define a geometric plane, as described in greater detail below with respect to FIGS. 2 and 3. Accordingly, the plurality of geometric shapes are configured to provide a scaffold structure defining a central void 112 therein.

Additionally, a geometric plane generally defined by the geometric shape 110A may be configured to pass within the central void 112. We have discovered that such a configuration of the distal-most geometric shape, e.g., geometric shape 110A, reduces the likelihood of the coil entering back into the vasculature during deployment, while encouraging continued deployment into a biological space or area, such as a space within an aneurysm. Furthermore, a geometric plane generally define by the geometric shape 110B may be configured to pass within the central void 112, as well. We have discovered that such a configuration of the geometric shape 110B, along with the tilted configuration of the geometric shape 110A, may cooperate to further encourage continued coil 100 deployment with the biological space.

Each of the plurality of geometric shapes 110 includes a dimension, a length if the geometric shape is more rectangular or a diameter if the geometric shape is more circular, for example, and one or more of dimensions of the plurality of geometric shapes 110 may be less than the remaining dimensions thereof to further encourage deployment into the confined area. For example, each of the plurality of geometric shapes 110 may be circular having a respective one of a plurality of diameters, and the diameter of the distal-most geometric shape 110A may be less than the diameters of the remaining ones of the plurality of diameters corresponding to the geometric shapes 110C-110F. Additionally, the diameter of the geometric shape 110B may be greater than the diameter of geometric shape 110A, but less than the remaining ones of the plurality of diameters corresponding to the geometric shapes 110C-110F. We have discovered that this configuration of the geometric shapes 110 may encourage the vaso-occlusive coil deployment to remain within the biological space of an aneurysm.

FIG. 2 depicts a side elevation view of the vaso-occlusive coil from a direction identified by an arrow A₂ of FIG. 1. As shown, the geometric shapes 110 are configured to define the central void 112 of the vaso-occlusive coil 100, which may be further defined by individual attributes of each of the geometric shapes 110. For example, each of the geometric shapes 110 may generally include one of a plurality of geometric planes, respectively, collectively referred to as geometric planes 110_{P}, and individually referred to as geometric planes 110A_{P}-110F_{P}. Each of the geometric planes 110_{P} are substantially, but not necessarily exactly, along a longitudinal axis of a respective one of the plurality of corresponding geometric shapes 110. As depicted in the exemplary device of FIG. 2, a geometric plane 110F_{P} of the geometric shape 110F is generally perpendicular to a geometric plane 110D_{P} of geometric shape 110D, which is generally perpendicular to a geometric plane 110C_{P} of geometric shape 110C. Geometric plane 110A_{P} of geometric shape 110A, however, may not be parallel, nor perpendicular, to the geometric planes 110C_{P}, 110D_{P}, 110F_{P}. Accordingly, geometric plane 110A_{P} of geometric shape 110A may pass within the central void 112 of the vaso-occlusive coil 100.

Additionally, each of the geometric shapes 110 of FIG. 2, since they are substantially circular in configuration, includes a respective one of a plurality of diameters, collectively referred to as diameters 110_{D}, and individually referred to as diameters 110A_{D}-110F_{D}. None, some, or all the diameters 110_{D} may be the same, or may be different. As depicted in FIG. 2, a diameter 110F_{D} may be similar to a diameter 110D_{D}, which may be similar to a diameter 110C_{D}. Preferably, a diameter 110A_{D} of the distal-most geometric shape 110A is less than the remaining diameters 110_{D} of the geometric shapes 110. Additionally, the geometric shape 110A may extend outside a scaffold structure generally defined by the remaining geometric shapes 110B-110F.

FIG. 3 depicts a side elevation view of the vaso-occlusive coil 100 from a direction identified by an arrow A₃ of FIG. 1. Once again, the geometric shapes 110 are configured to define the central void 112 of the vaso-occlusive coil 100. As depicted in the exemplary device of FIG. 3, the geometric plane 110F_{P} of the geometric shape 110F is generally perpendicular to a geometric plane 110E_{P} of geometric shape 110E, which is generally perpendicular to the geometric plane 110C_{P} of geometric shape 110C. Optionally, as depicted in FIG. 3, the geometric shape 110B may include a geometric plane 110B_{P} that is neither parallel, nor perpendicular, to the geometric planes 110C_{P}, 110E_{P}, 110F_{P}. Accordingly, as with the geometric plane 110A_{P}, geometric plane 110B_{P} of geometric shape 110B may pass within the central void 112 of the vaso-occlusive coil 100. As depicted in FIG. 3, a diameter 110F_{D} may be similar to a diameter 110E_{D}, which may be similar to a diameter 110C_{D}. A diameter 110B_{D} of geometric shape 110B may be less than the diameters 110C_{D}-110F_{D}, but greater than the diameter 110A_{D} of the distal-most geometric shape 110A. Additionally, as depicted, since both geometric planes 110A_{P} and 110B_{P} are neither parallel, nor perpendicular, to the remaining geometric planes 110C_{P}-110F_{P}, a portion of the coil 100 corresponding to a portion of geometric shape 110A may pass through the geometric plane 110B_{P}. Such a configuration may further encourage desired deployment of the vaso-occlusive coil 100 within the biological space of an aneurysm.

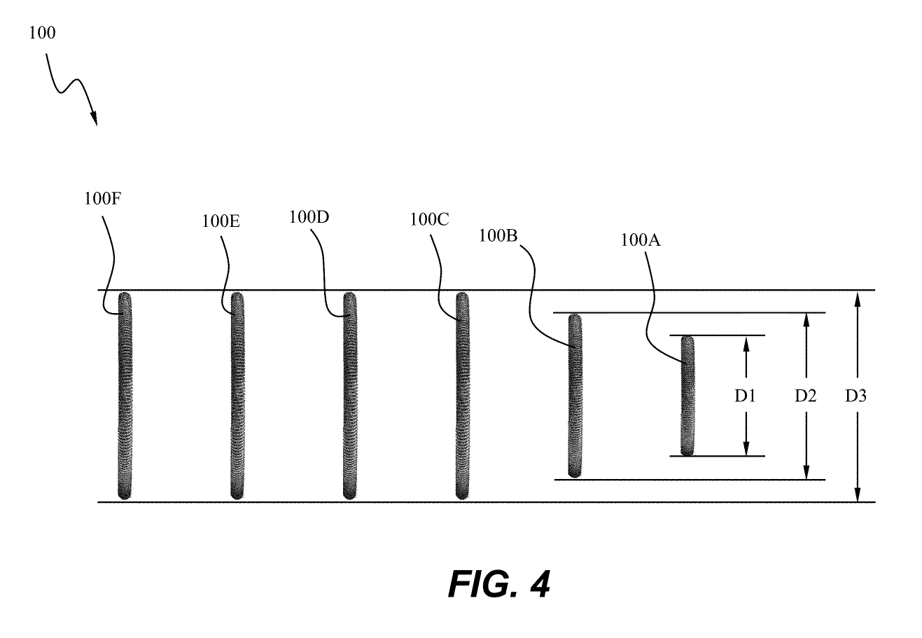

Turning to FIG. 4, exemplary values of the diameters 110D are depicted relative to each other. For example, with reference to the exemplary vaso-occlusive coil 100 of FIG. 1, the distal-most geometric shape 100A may have a first diameter D1, the next distal-most geometric shape 100B may have a second diameter D2, and the remaining geometric shapes 100C-100F may have a third diameter D3. As depicted, the diameter D2 may be less than the diameter D3, but greater than diameter D1. We have discovered that a diameter D1 of geometric shape 100A being less than the remaining diameters D2, D3 encourages the deployment of the vaso-occlusive coil 100 within an aneurysm. Additionally, we have also discovered that providing a second distal-most diameter that is greater than the distal-most diameter D1, but less than the remaining diameters D3, may help to further encourage the deployment of the vaso-occlusive coil within the aneurysm. While the geometric shapes 100C-100F are depicted as having similar diameters D3, each of the geometric shapes 100C-100F may include the same or differing diameters than other ones of the diameters of the geometric shapes 100C-100F.

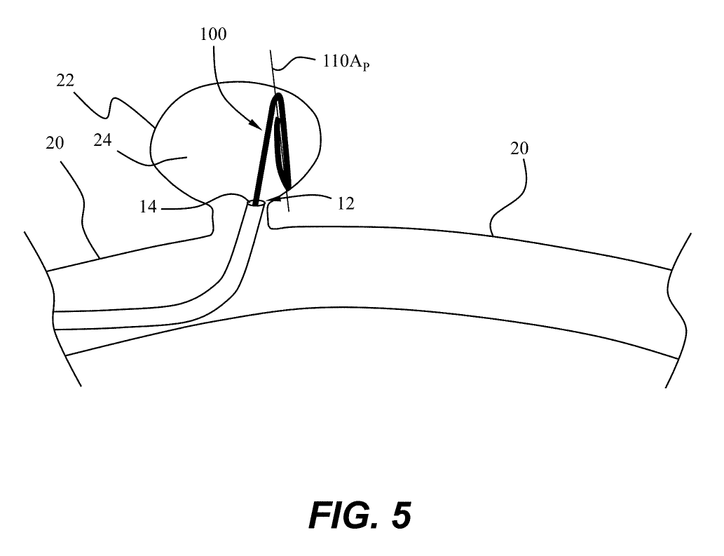

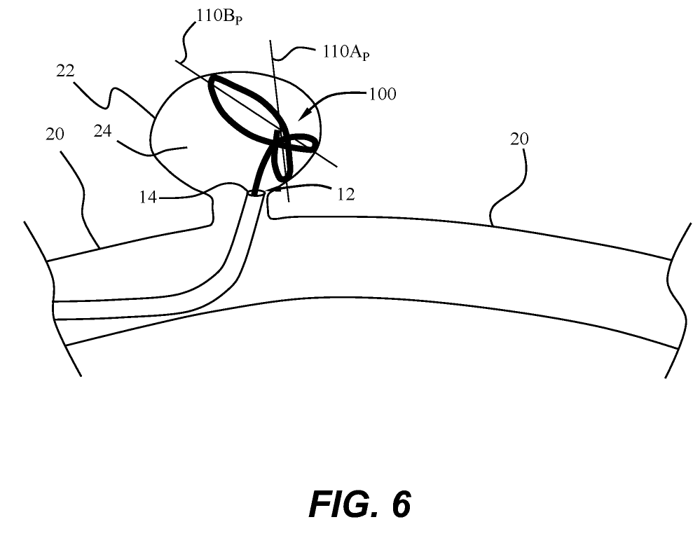

Turning to FIGS. 5 and 6, deployment of vaso-occlusive coil 100 within an aneurysm 22, e.g., within a biological space 24 of an aneurysm 22 includes advancing catheter 10 through vasculature 20 until the distal opening 14 of lumen 12 is at or near the opening of the aneurysm 22. Once the catheter 10 is positioned, the deployment of the vaso-occlusive coil 100 may start with the deployment of the distal-most geometric shape 110A. Due to the nature of the transition between the geometric shape 110A and geometric shape 110B, the geometric shape 110A, and corresponding geometric plane 110A_{P}, may be positioned within the aneurysm 22 as generally depicted in FIG. 5, encouraging further deployment of the coil 100 within the aneurysm 22.

Turning specifically to FIG. 6, the vaso-occlusive coil 100 may be further deployed within the aneurysm 22, where the geometric shape 110B may be positioned relative to geometric shape 110A as generally depicted in FIG. 6. As shown, with the deployment of geometric plane 110B, the geometric plane 110B_{P} of geometric shape 110B forms a relationship with geometric plane 110A_{P} of geometric shape 110A. We have found that this initial relationship between geometric planes 110A_{P} and 110B_{P} encourages further deployment of the remaining geometric shapes 110C-110F within the void of an aneurysm, while discouraging the deployment of part of the vaso-occlusive coil 100 back into the vasculature 20 of the patient.

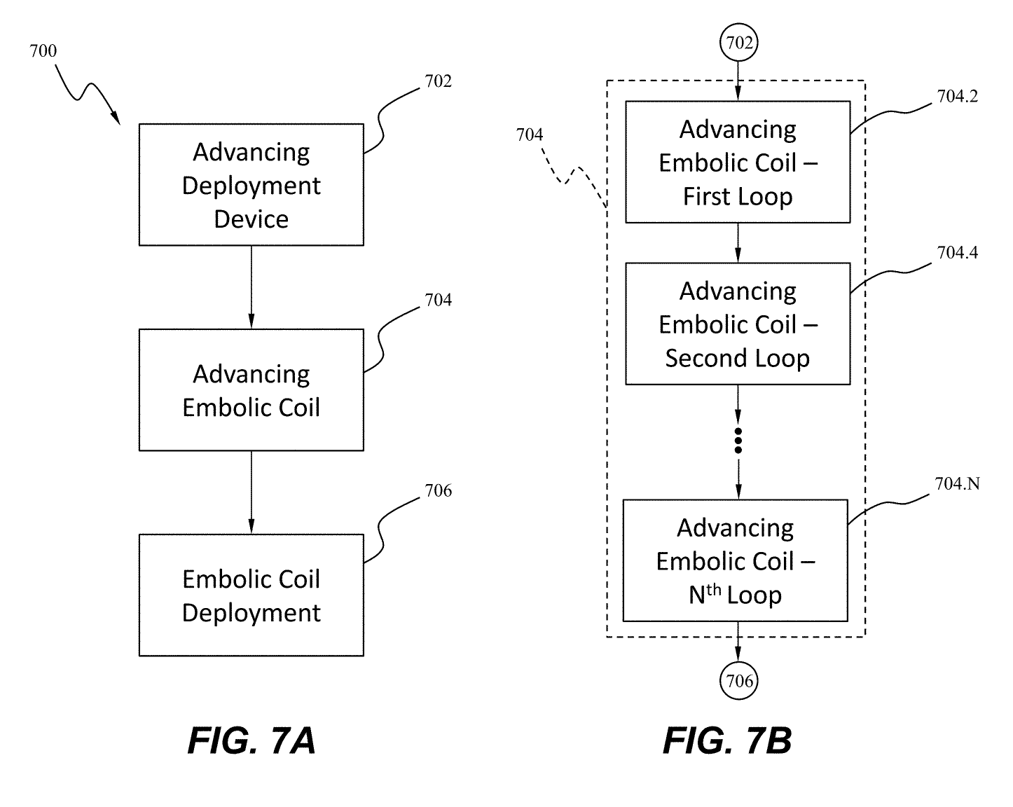

Turning to FIG. 7A, an exemplary method 700 includes an initial step 702 of advancing a deployment device to a target tissue site, e.g., the location of an aneurysm. Once the deployment device is positioned, an embolic coil as described or contemplated herein may then be advanced into the aneurysm in a step 704. Once the embolic coil is fully advanced within the aneurysm, the embolic coil may be severed, or otherwise detached, from the deployment device, completing deployment of the embolic coil in a step 706. With reference to FIG. 7B, the step 704 of advancing the embolic coil may include a first step 704.1 of advancing a first geometric shape, e.g. a first geometric loop, within the aneurysm, and a second step 704.2 of advancing a second geometric shape, e.g., a second geometric loop, within the aneurysm. The step 704 may further include a step 704.N of advancing an Nth geometric shape, e.g., Nth geometric loop, within the aneurysm, where N is greater than 2. Accordingly, the embolic coil as deployed may include less geometric shapes than a total number of geometric shapes depicted in embolic coil 100 of FIG. 1.

As noted previously, the forgoing descriptions of the specific embodiments are presented for purposes of illustration and description, and are not intended to be exhaustive or to be limited to the precise forms disclosed, as many modifications and variations are possible in view of the above teachings. The embodiments were chosen and described to illustrate and explain principles and practical applications, to thereby enable those skilled in the art to best utilize the various embodiments thereof as suited to the particular use contemplated.

1. A vaso-occlusive coil having a primary shape when confined within a lumen of a deployment device, and a secondary shape when unconfined, the secondary shape including a plurality of geometric shapes, a first of the plurality of geometric shapes having a geometric plane along its longitudinal axis, remaining ones of the plurality of geometric shapes cooperating to form a central void, the geometric plane of the first of the plurality of geometric shapes passing through the central void.

2. The vaso-occlusive coil of claim 1, wherein a portion of the first of the plurality of geometric shapes is positioned within the central void.

3. The vaso-occlusive coil of claim 1, wherein the primary shape is substantially straight, the vaso-occlusive coil transitioning from the primary shape to the secondary shape as the vaso-occlusive coil is deployed from the lumen of the deployment device.

4. The vaso-occlusive coil of claim 1, wherein each of the plurality of geometric shapes is selected from a group of shapes including: a circular shape, a rectangular shape, and a triangular shape.

5. The vaso-occlusive coil of claim 1, wherein a length of the first of the plurality of geometric shapes is less than a length of each of the remaining ones of the plurality of geometric shapes.

6. The vaso-occlusive coil of claim 1, wherein a length of the coil extends along the plurality of geometric shapes, the first of the plurality of geometric shapes being a distal-most geometric shape along the length of the coil.

7. The vaso-occlusive coil of claim 1, wherein a second of the plurality of geometric shapes includes a geometric plane along its longitudinal axis, the geometric plane of the second of the plurality of geometric shapes passing through the central void.

8. The vaso-occlusive coil of claim 7, wherein a length of coil extends along the plurality of geometric shapes, the first of the plurality of geometric shapes being adjacent to the second of the plurality of geometric shapes along the length of the coil.

9. The vaso-occlusive coil of claim 8, wherein the first of the plurality of geometric shapes is a distal-most geometric shape along the length of the coil.

10. The vaso-occlusive coil of claim 7, wherein a length of the second of the plurality of geometric shapes is less than a length of each of the remaining ones of the plurality of geometric shapes.

11. The vaso-occlusive coil of claim 10, wherein the length of the second of the plurality of geometric shapes is greater than a length of the first of the plurality of geometric shapes.

12. The vaso-occlusive coil of claim 1, wherein each of the plurality of geometric shapes includes a respective one of a plurality of geometric planes along its corresponding longitudinal axis, the geometric plane of the first of the plurality of geometric planes being nonparallel with respect to each of the remaining ones of the plurality of geometric planes.

13. The vaso-occlusive coil of claim 1, wherein each of the plurality of geometric shapes includes a respective one of a plurality of geometric planes along its corresponding longitudinal axis, the geometric plane of the first of the plurality of geometric planes being non-perpendicular with respect to each of the remaining ones of the plurality of geometric planes.

14. The vaso-occlusive coil of claim 1, wherein each of the plurality of geometric shapes includes a respective one of a plurality of geometric planes along its corresponding longitudinal axis, the geometric plane of each of the first of the plurality of geometric planes and a second of the plurality of geometric planes being nonparallel with respect to each of the remaining ones of the plurality of geometric planes.

15. The vaso-occlusive coil of claim 1, wherein each of the plurality of geometric shapes includes a respective one of a plurality of geometric planes along its corresponding longitudinal axis, the geometric plane of each of the first of the plurality of geometric planes and a second of the plurality of geometric planes being non-perpendicular with respect to each of the remaining ones of the plurality of geometric planes.

16. The vaso-occlusive coil of claim 1, wherein a portion of the first of the plurality of geometric shapes passes through the geometric plane of a second of the plurality of geometric shapes, the second of the plurality of geometric shapes being adjacent to the first of the plurality of geometric shapes along a length of the vaso-occlusive coil.

17. A method, comprising:
providing an vaso-occlusive coil having a primary shape and a secondary shape, the secondary shape including a plurality of geometric shapes, a first of the plurality of geometric shapes having a geometric plane along its longitudinal axis, remaining ones of the plurality of geometric shapes cooperating to form a central void, the geometric plane of the first of the plurality of geometric shapes passing into the central void;
advancing a deployment device to a target tissue site, the deployment device having a lumen there through;
advancing a portion of the vaso-occlusive coil through the lumen of the deployment device.

18. The method of claim 17, wherein advancing a portion of the vaso-occlusive coil through the lumen of the deployment device includes advancing the first of the plurality of geometric shapes through the lumen of the deployment device, the geometric plane of the first of the plurality of geometric shapes forming an acute angle with a length of the vaso-occlusive coil immediately adjacent to the first of the plurality of geometric shapes.

19. The method of claim 18, wherein advancing a portion of the vaso-occlusive coil through the lumen of the deployment device includes advancing a second of the plurality of geometric shapes through the lumen of the deployment device.
